(19) United States
(12) Patent Application Publication
(10) Pub. No.: US 2015/0276759 A1
(43) Pub. Date: Oct. 1, 2015

(54) METHOD AND APPARATUS FOR DETERMINING A MAMMAL'S EXPOSURE TO CHEMICAL OR BIOLOGICAL AGENTS

(71) Applicant: Epicgenetics LLC, Los Angeles, CA (US)

(72) Inventors: Bruce S. Gillis, Beverly Hills, CA (US); Igor Mikhailovich Gavin, Bolingbrook, IL (US)

(21) Appl. No.: 14/607,965

(22) Filed: Jan. 28, 2015

Related U.S. Application Data
(63) Continuation of application No. 13/204,105, filed on Aug. 5, 2011, now abandoned.

(51) Int. Cl. G01N 33/68

(52) U.S. Cl. CPC G01N 33/6863; G01N 2570/00

(57) ABSTRACT

Cellular immunologic methods are disclosed for determining human health effects of exposures to environmental molds and toxins. In one embodiment, a method for assessing a patient's exposure to a mold strain is provided. The method comprises measuring cytokine production in at least the peripheral blood mononuclear cells of a mammal suspected of being exposed to mold or other toxin and determining if there is a decreased production of cytokines in the mammal suspected of being exposed to mold or other toxin when compared to the production of cytokines produced in a mammal not suspected of being exposed to mold or other toxin. Other methods, including methods of of diagnosing respiratory disorders, including asthma, are also disclosed.

### REFERENCE TO BIBLIOGRAPHY

The bibliography lists articles referenced during the course of the study conducted regarding immunosuppressive effects on mold on humans, all of which are incorporated herein by reference.

### BACKGROUND

1. Technical Field

The present disclosure relates generally to measuring the human health effects of exposures to environmental toxins and, more particularly, to measuring the expression and inhibition of cytokines when humans are exposed to mold, mycotoxins, and other toxins.

2. Description of Related Art

Mold has been suspected as an adverse cause of health in indoor environments since it was originally mentioned in Leviticus (13:47-50 and 14:45) in the Old Testament. A more recent set of concerns has appeared in response to the respiratory symptoms registered by individuals who have either lived or worked in mold-contaminated environments, usually as a result of excess dampness. A debate has raged among researchers as to whether mold-contaminated indoor environments lead to acute, chronic, or permanent human health complications. Much of this debate is based upon claimed economic interests, some of which appear to be driven by potentially self-serving interests, to limit the financial repercussions of any cause-and-effect relationships between mold exposures and human health problems.

Misinformation has also appeared about identifying what a sufficient level of mold contamination must exist for health consequences to appear. The depth and breadth of the debate was summarized in the 2004 Institute of Medicine (IOM) publication, Damp Indoor Spaces and Health [Institute of Medicine (U.S.) CoDISaH. Damp Indoor Spaces and Health. 2004.]. The IOM authors found sufficient evidence that excessive moisture and mold are associated with the presence of respiratory symptoms. Yet other studies suggested that mold and moisture problems might increase the risk of developing atopic symptoms and allergic sensitization not only to mold, but also to other common inhaled allergens, and increase the risk of secondary infections [Jacob B, Ritz B, Gehring U, Koch A, Bischof W, Wichmann H E, Heinrich J. Indoor exposure to molds and allergic sensitization. Environ Health Perspect 2002; 110: 647-653 and Garrett M H, Rayment P R, Hooper M A, Abramson M J, Hooper B M. Indoor airborne fungal spores, house dampness and associations with environmental factors and respiratory health in children. Clin Exp Allergy 1998; 28: 459-467.].

A number of studies found a significant correlation between exposures to a damp indoor environment and the presence of various upper and lower respiratory symptoms [Hope A P, Simon R A. Excess dampness and mold growth in homes: an evidence-based review of the aeroirritant effect and its potential causes. Allergy Asthma Proc 2007;28: 262-270; Bornehag C G, Sundell J, Bonini S, Custovic A, Malmberg P, Skerfving S, Sigsgaard T, Verhoeff A. Dampness in buildings as a risk factor for health effects, EUROEXPO: a multidisciplinary review of the literature (1998-2000) on dampness and mite exposure in buildings and health effects. Indoor Air 2004;14: 243-257; Bornehag C G, Blomquist G, Gyntelberg F, Jarvholm B, Malmberg P, Nordvall L, Nielsen A, Pershagen G, Sundell J. Dampness in buildings and health. Nordic interdisciplinary review of the scientific evidence on associations between exposure to “dampness” in buildings and health effects (NORDDAMP). Indoor Air 2001;11: 72-86; and Koskinen O M, Husman T M, Meklin T M, Nevalainen A I. The relationship between moisture or mold observations in houses and the state of health of their occupants. Eur Respir J 1999;14: 1363-1367.]. Many of these have included surveys based purely upon subjective complaints, which were either the result of self-reporting where certain biases were apparent, or they arose in studies by particular investigators who failed to eliminate chance or bias [Engman L H, Bornehag C G, Sundell J. How valid are parents' questionnaire responses regarding building characteristics, mouldy odour, and signs of moisture problems in Swedish homes? Scand J Public Health 2007; 35: 125- 132; Bornehag C G, Sundell J, Sigsgaard T, Janson S. Potential self-selection bias in a nested case-control study on indoor environmental factors and their association with asthma and allergic symptoms among pre-school children. Scand J Public Health 2006; 34: 534-543; Dales R E, Miller D, McMullen E. Indoor air quality and health: validity and determinants of reported home dampness and moulds. Int J Epidemiol 1997; 26: 120-125; and Strachan DP. Damp housing and childhood asthma: validation of reporting of symptoms. Bmj 1988; 297: 1223-1226.].

To eliminate chance or bias in associating clinical symptoms with exposure to mold, the evaluation should be done based on the results of medical examination and other objective criteria. Previous studies of mold-exposed patients were based on clinical histories, physical examinations, pulmonary function analyses and allergy analyses. However, common clinical tests fail to establish a definitive link between chronic exposures and adverse health effects. For example, widely used immunological tests, such as IgE measurements or skin prick tests, were shown to be poor markers of mold exposures [Hyvarinen A, Husman T, Laitinen S, Meklin T, Taskinen T, Korppi M, Nevalainen A. Microbial exposure and mold-specific serum IgG levels among children with respiratory symptoms in 2 school buildings. Arch Environ Health 2003; 58: 275-283; Immonen J, Laitinen S, Taskinen T, Pekkanen J, Nevalainen A, Korppi M. Mould-specific immunoglobulin G antibodies in students from moisture- and mould-damaged schools: a 3-year follow-up study. Pediatr Allergy Immunol 2002; 13: 125-128; Taskinen T M, Laitinen S, Nevalainen A, Vepsalainen A, Meklin T, Reiman M, Korppi M, Husman T. Immunoglobulin G antibodies to moulds in school- children from moisture problem schools. Allergy 2002; 57: 9-16; Immonen J, Meklin T, Taskinen T, Nevalainen A, Korppi M. Skin-prick test findings in students from moisture- and mould-damaged schools: a 3-year follow-up study. Pediatr Allergy Immunol 2001; 12: 87-94; and Taskinen T, Meklin T, Nousiainen M, Husman T, Nevalainen A, Korppi M. Moisture and mould problems in schools and respiratory manifestations in schoolchildren: clinical and skin test findings. Acta Paediatr 1997; 86: 1181-1187.].

The body of evidence strongly suggests that exposure to mold induces immune responses both in animals and in humans. For example, a number of studies demonstrated that challenges to mold activate inflammatory cells and increase cytokine production in vivo [Stark H, Roponen M, Purokivi M, Randell J, Tukiainen H, Hirvonen M R. Aspergillus fumigatus challenge increases cytokine levels in nasal lavage fluid. Inhal Toxicol 2006; 18: 1033-1039; Havaux X, Zeine A, Dits A, Denis O. A new mouse model of lung allergy induced by the spores of Alternaria alternata and Cladosporium herbarum molds. Clint Exp Immunol 2005;139: 179-188; Hudson B, Flemming J, Sun G, Rand T G. Comparison of immunomodulator mRNA and protein expression in the lungs of Stachybotrys chartarum spore-exposed mice. J Toxicol Environ Health A 2005;68: 1321-1335; Yike I, Rand T G, Dearborn D G. Acute inflammatory responses to Stachybotrys chartarum in the lungs of infant rats: time course and possible mechanisms. Toxicol Sci 2005; 84: 408-417; Purokivi M K, Hirvonen M R, Randell J T, Roponen M H, Meklin T M, Nevalainen A L, Husman T M, Tukiainen H O. Changes in pro-inflammatory cytokines in association with exposure to moisture-damaged building microbes. Eur Respir J2001; 18: 951-958; and Hirvonen M R, Ruotsalainen M, Roponen M, Hyvarinen A, Husman T, Kosma V M, Komulainen H, Savolainen K, Nevalainen A. Nitric oxide and proinflammatory cytokines in nasal lavage fluid associated with symptoms and exposure to moldy building microbes. Am J Respir Crit Care Med 1999; 160: 1943-1946.] and in cell cultures [Chiu L L, Perng D W, Yu C H, Su S N, Chow L P. Mold allergen, pen C 13, induces IL-8 expression in human airway epithelial cells by activating protease-activated receptor 1 and 2. J Immunol 2007; 178: 5237-5244; Shalit I, Halperin D, Haite D, Levitov A, Romano J. Osherov N, Fabian I. Anti-inflammatory effects of moxifloxacin on IL-8, IL-1beta and TNF-alpha secretion and NFkappaB and MAP-kinase activation in human monocytes stimulated with Aspergillus fumigatus. J Antimicrob Chemother 2006; 57: 230-235; Inoue Y, Matsuwaki Y, Shin S H, Ponikau J U, Kita H. Nonpathogenic, environmental fungi induce activation and degranulation of human eosinophils. J Immunol 2005; 175: 5439-5447; Overland G, Stuestol J F, Dahle M K, Myhre A E, Netea M G, Verweij P, Yndestad A, Aukrust P, Kullberg B J, Warris A, Wang J E, Aasen A O. Cytokine responses to fungal pathogens in Kupffer Cells are Toll-like receptor 4 independent and mediated by tyrosine kinases. Scand J Immunol 2005; 62: 148-154; Huttunen K, Hyvarinen A, Nevalainen A, Komulainen H, Hirvonen M R. Production of proinflammatory mediators by indoor air bacteria and fungal spores in mouse and human cell lines. Environ Health Perspect 2003; 111: 85-92; Murtoniemi T, Nevalainen A, Suutari M, Hirvonen M R. Effect of liner and core materials of plasterboard on microbial growth, spore-induced inflammatory responses, and cytotoxicity in macrophages. Inhal Toxicol 2002; 14: 1087- 1101; Murtoniemi T, Nevalainen A, Suutari M, Toivola M, Komulainen H, Hirvonen M R. Induction of cytotoxicity and production of inflammatory mediators in raw264.7 macrophages by spores grown on six different plasterboards. Inhal Toxicol 2001; 13: 233-247; Kauffman H F, Tomee J F, van de Riet M A, Timmerman A J, Borger P. Protease-dependent activation of epithelial cells by fungal allergens leads to morphologic changes and cytokine production. J Allergy Clin Immunol 2000; 105: 1185-1193; Rongrungruang Y, Levitz S M. Interactions of Penicillium marneffei with human leukocytes in vitro. Infect Immun 1999;67: 4732-4736; Shahan T A, Sorenson W G, Paulauskis J D, Morey R, Lewis D M. Concentration-and time-dependent upregulation and release of the cytokines MIP-2, KC, TNF, and MIP-1alpha in rat alveolar macrophages by fungal spores implicated in airway inflammation. Am J Respir Cell Mod Biol 1998; 18: 435-440; and Grazziutti M L, Rex J H, Cowart R E, Anaissie E J, Ford A, Savary C A. Aspergillus fumigatus conidia induce a Th1-type cytokine response. J Infect Dis 1997; 176: 1579-1583.]. However, the effects of prior chronic exposures to molds on respiratory and immune responses were poorly investigated.

### SUMMARY

One aspect of the present disclosure is to provide methods and related systems to identify the most accurate means of documenting human health consequences from exposures to chemical or biological agents.

In one embodiment of the present disclosure, a method of determining whether a mammal has been exposed to a chemical or biological agent is provided. The method includes one or more aspects of (a) exposing the isolated mammalian cells from one or more mammals exposed to and one or more mammals not exposed to a non-toxic amount of a chemical or biological test agent, where the chemical or biological test agent is effective to induce a measurable response in the level of a plurality of biomarkers in the isolated mammalian cells from exposed mammals and is not substantially lethal to the survival of the cells, (b) determining one or more cellular responses to the chemical or biological agent by measuring the level of a plurality of biomarkers of the isolated mammalian cells from the exposed and not exposed mammals, (c) comparing the cellular responses of the mammals that were exposed to the cellular responses of the mammals not exposed to the chemical or biological agent by determining the difference in the level of the plurality of biomarkers, (d) generating biomarker profile for the chemical or biological agent based on the change in the level of a plurality of a subset of the biomarkers, wherein the subset of biomarkers is sufficient to establish a statistically significant correlation between exposure to the agent and the biomarker profile, (e) isolating mammalian cells from a mammal who is suspected of being exposed to the chemical or biological agent, (f) determining one or more cellular responses to the chemical or biological agent of the cells by measuring the presence or level of a plurality of biomarkers of the mammalian cells from the mammal suspected of being exposed to the chemical or biological agent, and (g) determining whether the mammal has been exposed to the chemical or biological agent by determining whether there is a statistically significant correlation between the measured presence or level of the plurality of biomarkers and a biomarker profile generated by measuring the presence or level of a plurality of biomarkers of the mammalian cells from a mammal exposed to the chemical or biological agent.

In an aspect of at least one embodiment of the present disclosure, the method includes mammalian cells selected from the group consisting of peripheral blood mononuclear cells, lymphocytes, leukocytes, dendritic cells, epithelial cells, neural cells, skin cells and other known mammalian cells that would be appropriate.

In yet another aspect of at least one embodiment of the present disclosure, the cellular responses may be one or more selected from the group consisting of cell proliferation, cell function, cell death, gene expression, protein expression, protein modification, RNA modification, DNA modification, protein function, RNA function, expression of metabolites, protein transfer, RNA transfer, secretion of proteins, and secretion of metabolites.

In yet another aspect of at least one embodiment of the present disclosure, the cellular responses may be extracellular expressions of cytokines.

In yet another aspect of at least one embodiment of the present disclosure, the biomarkers may be one or more selected from the group consisting of nucleic acids, proteins, peptides, metabolites, cytokines, chemokines, growth factors and other known biomarkers.

In yet another aspect of at least one embodiment of the present disclosure, the biomarkers may be one or more cytokines selected from the group consisting of GM-CSF, IL-1β, IL-6, IL-8, TNF-α, IFN-γ, IL-2, IL-4, IL-5, IL-10, MIP-1α, MIP-1β, MCP-1, Eotaxin, and RANTES.

In yet another aspect of at least one embodiment of the present disclosure, the chemical or biological test agent is selected from the group consisting of industrial chemicals, environmental pollutants, biological toxins, mold, allergens, and antigens.

In yet another aspect of at least one embodiment of the present disclosure, the method is used to detect exposure to, or inhalation of, mycotoxins.

In yet another aspect of at least one embodiment of the present disclosure, the agent may be mold selected from the group consisting of Aspergillus niger, Cladosporium herbarum, and Penicillium chrysogenum.

In yet another aspect of at least one embodiment of the present disclosure, the method includes one or more aspects of collecting blood samples from patients, isolating peripheral blood mononuclear cells from blood samples collected from patients, culturing those peripheral blood mononuclear cells in the presence of a particular strain of mold, culturing the peripheral blood mononuclear cells in the presence of phytohemagglutinin, determining the cellular responses of the peripheral blood mononuclear cells by measuring the extracellular expressions of particular cytokines, determining the cellular responses to phytohemagglutinin of the peripheral blood mononuclear cells by measuring the extracellular expressions of cytokines, comparing the cellular responses to mold of the peripheral blood mononuclear cells to cellular responses to phytohemagglutinin of the peripheral blood mononuclear cells.

In another embodiment of the present disclosure, a method of determining whether a mammal has been exposed to a chemical or biological agent is provided. The method includes one or more aspects of (a) isolating mammalian cells from a mammal who is suspected of being exposed to the chemical or biological agent, (b) determining one or more cellular responses to the chemical or biological agent of the cells by measuring the presence or level of a plurality of biomarkers of the mammalian cells from the mammal suspected of being exposed to the chemical or biological agent, and (c) determining whether the mammal has been exposed to the chemical or biological agent by determining whether there is a statistically significant correlation between the measured presence or level of the plurality of biomarkers and a biomarker profile generated by measuring the presence or level of a plurality of biomarkers of the mammalian cells from a mammal exposed to the chemical or biological agent.

In yet another aspect of at least one embodiment of the present disclosure, the mammalian cells are selected from the group consisting of peripheral blood mononuclear cells, lymphocytes, leukocytes, dendritic cells, epithelial cells, neural cells, and skin cells.

In yet another aspect of at least one embodiment of the present disclosure, the cellular responses may be one or more selected from the group consisting of cell proliferation, cell death, cell function, gene expression, protein expression, protein modification, RNA modification, DNA modification, protein function, RNA function, expression of metabolites, protein transfer, RNA transfer, secretion of proteins, and secretion of metabolites.

In yet another aspect of at least one embodiment of the present disclosure, the cellular responses may be extracellular expressions of cytokines

In yet another aspect of at least one embodiment of the present disclosure, the biomarkers may be one or more selected from the group consisting of nucleic acids, proteins, peptides, metabolites, cytokines, chemokines, and growth factors.

In yet another aspect of at least one embodiment of the present disclosure, the biomarkers may be one or more cytokines selected from the group consisting of GM-CSF, IFN-β, ILA-β, IL-6, IL-8, IL-10, TNF-α, MIP1-β, MCP-1, Eotaxin, MIP1-α and RANTES.

In yet another aspect of at least one embodiment of the present disclosure, the chemical or biological test agent is selected from the group consisting of industrial chemicals, environmental pollutants, biological toxins, mold, allergens, and antigens.

In yet another aspect of at least one embodiment of the present disclosure, the method is used to detect exposure to, or inhalation of, mycotoxins.

In yet another aspect of at least one embodiment of the present disclosure, the agent may be mold selected from the group consisting of Aspergillus niger, Cladosporium herbarum, and Penicillium chrysogenum.

In yet another aspect of at least one embodiment of the present disclosure, the cellular responses to the chemical or biological agent are compared to the cellular responses to a reference agent.

In yet another aspect of at least one embodiment of the present disclosure, the reference agent may be selected from the group consisting of lectins and their derivatives, phorbol esters and their derivatives, ionomycinand its derivatives, lipopolysaccharides and their derivatives, antibodies, and cytokines and their derivatives.

In another aspect of at least one embodiment of the present disclosure, the reference agent may be phytohemagglutinin.

In yet another aspect of at least one embodiment of the present disclosure, a decreased production of GM-CSF, IL1-β, IL-6, IL-8, IL-10, MIP1-β, MCP-1, Eotaxin, MIP1-α and RANTES indicates that the individual has been exposed to P. chrysogenum.

In yet another aspect of at least one embodiment of the present disclosure, a decreased production of GM-CSF, IL1-β, IL-6, IL-8, MIP1-β, Eotaxin, MIP1-α and RANTES indicates that the individual has been exposed to C. herbarum.

In yet another aspect of at least one embodiment of the present disclosure, a decreased production of IL-10, MIP1-β and Eotaxin indicates that the individual has been exposed to A. niger. In yet another embodiment of the present disclosure, a diagnostic kit for measuring a mammal's exposure to a chemical or biological test agent is provided. The kit includes one or more aspects of (a) a chemical or biological test agent, (b) a mammalian cell culture medium, (c) a balanced salt solution and (d) a reference agent. It should be appreciate that many items in the kits disclosed herein are general lab equipment and supplies and can be purchased separately.

It should also be appreciate that using various mammalian cell culture media and balanced salt solutions is part of the methods and kits disclosed herein and would be obvious to one of ordinary skill. In at least one embodiment, the kit would include mold cells and PHA. It should also be appreciate that the present disclosure includes a cell isolation kit that may be included as part of the diagnostic kit described and claimed herein or sold separately.

In yet another aspect of at least one embodiment of the present disclosure, the chemical or biological test agent is selected from the group consisting of industrial chemicals, environmental pollutants, biological toxins, mold, allergens, and antigens.

In another aspect of at least one embodiment of the present disclosure, the mammalian cell culture medium may be RPMI 1640 medium, supplemented with L-glutamine, antibiotics, and 10% fetal bovine albumin.

In yet another aspect of at least one embodiment of the present disclosure, the balanced salt solution may be PBS, DPBS, HBSS, EBSS or their derivatives.

In another aspect of at least one embodiment of the present disclosure, the chemical or biological test agent may be mold cells selected from one or more of the group consisting of Aspergillus niger, Cladosporium herbarum, and Penicillium chrysogenum.

In yet another aspect of at least one embodiment of the present disclosure, the reference agent may be selected from one or more of the group consisting of lectins and their derivatives, phorbol esters and their derivatives, ionomycin, and its derivatives, lipopolysaccharides and their derivatives, antibodies, and cytokines and their derivatives.

In another aspect of at least one embodiment of the present disclosure, the reference agent may be phytohemagglutinin.

In yet another embodiment of the present disclosure, a kit for evaluating an individual afflicted with or at risk of developing FM is provided. The kit includes a multiplex of reagents for determining one or more cytokines

In one embodiment, the kit includes a multiplex of antibody-coupled beads, each antibody being specific for each of the cytokines to be determined in the assay, such as but not limited, to ELISA, tetramer assay, ELISPOT, Fluorspot, immuno-diffusion, etc. The kit also provides a detection system. Non-limiting examples of detection systems include one or more radio-labeled, fluorescent-labeled, enzyme-labeled secondary antibody or antibodies. Examples of fluorescent-labels are well known in the art and they include fluorescein, Texas red, rhodamine, etc. The bound sample can be analyzed using a flow cytometer, a fluorescent microscope, a ELISA reader, Luminex xMAP bead array technology, or Bio-Plex 200 fluorescence bead reader, etc.

For the purpose of the present disclosure, the following symbol “/” is intended to mean a ratio between an expression in front of the symbol and another expression after the symbol. These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings disclose illustrative embodiments and detail an aspect of the present disclosure. They do not set forth all embodiments. Other embodiments may be used in addition or instead. Details that may be apparent or unnecessary may be omitted to save space or for more effective illustration. Conversely, some embodiments may be practiced without all of the details that are disclosed. When the same numeral appears in different drawings, it is intended to refer to the same or like components or steps.

FIG. 1 shows assays that demonstrate the extracellular expression of cytokines in peripheral blood mononuclear cell cultures of healthy volunteers.

FIG. 2 is a graph showing the cytokine expression profiles in individual mold challenges.

FIG. 3 shows assays that demonstrate the inhibition of cytokine responses to mold challenges in patient samples.

FIG. 4 is a graph showing the correlation of an inhibition of cytokine response in patients with a positive methacholine challenge pulmonary test response.

FIG. 5 is a table summarizing the statistically significant differences in cytokine expression.

### DETAILED DESCRIPTION OF ILLUSTRATIVE EMBODIMENTS

Illustrative embodiments are now discussed. Other embodiments may be used in addition or instead. Details that may be apparent or unnecessary may be omitted to save space or for a more effective presentation. Conversely, some embodiments may be practiced without all of the details that are disclosed.

A number of negative health effects are associated with mold exposure, especially those affecting the immune and respiratory systems. In an attempt to better understand the health risks associated with chronic exposures to mold, a study was conducted of 11 office workers whose office building was contaminated by various concentrations of three molds: Aspergillus niger, Cladosporium herbarum, and Penicillim chrysogenum. Researchers analyzed and categorized patient symptoms, physical examination findings, static pulmonary function testing, methacholine challenge pulmonary testing, pulmonary exercise testing, total IgE, mold-specific radioallergosorbent (RAST) testing, and immunologic challenges to the molds.

Individual immunological responses to mold were investigated by measuring the production of cytokines, proteins and all other cell-released and mediated substances in peripheral blood mononuclear cell (PMBC) cultures challenged to mold cells. Researchers also determined how previous exposures to mold affected immune cells by comparing immunologic responses of patients with those of a matched control group based upon age, gender, ethnicity, and a lack of related symptoms from the same geographic location and who had no history of known exposures to mold contamination either in their worksites or their residences. Researchers also answered whether or not specific concentrations of indoor molds must be in existence in order to see adverse human health effects.

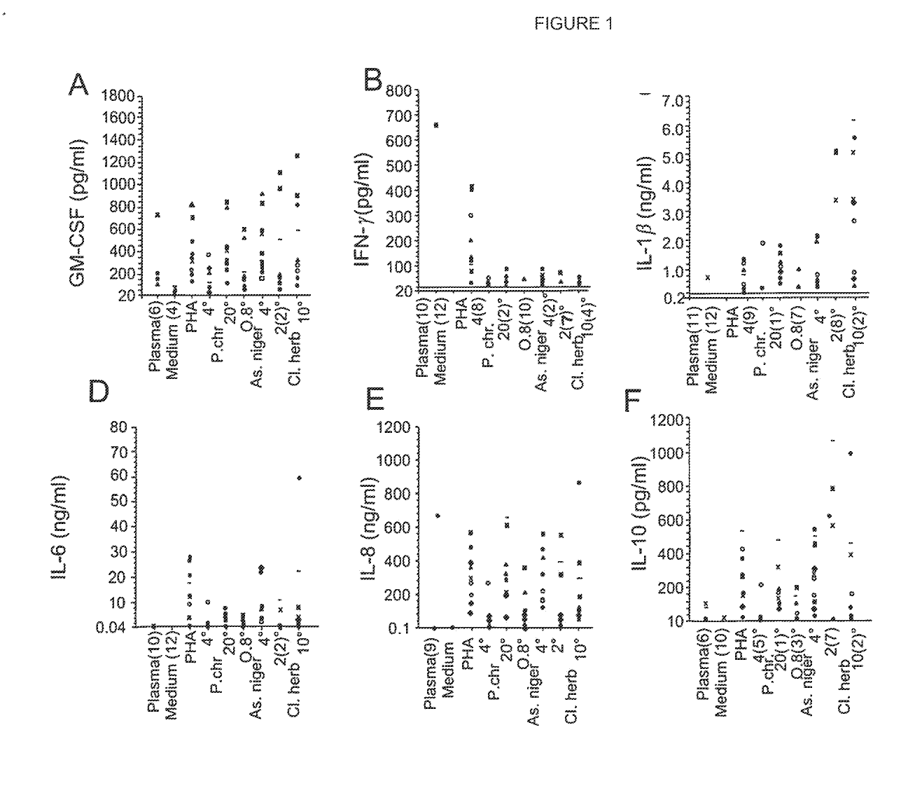

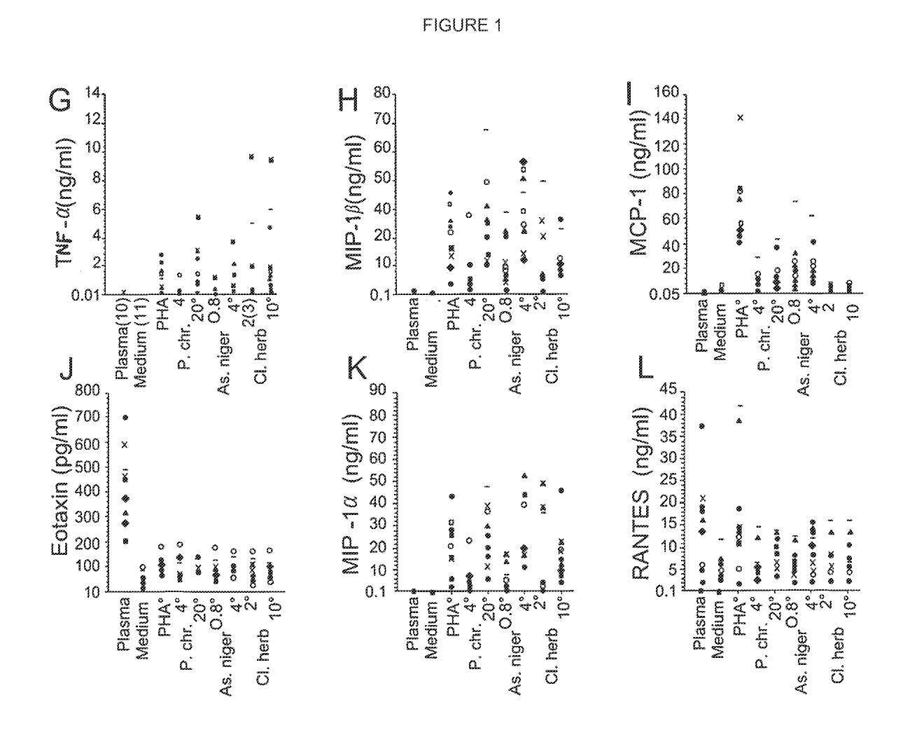

Referring to FIGS. 1-4, an extensive study was undertaken in which the cytokine levels of mold-exposed patients were compared with those of control subjects.

Mold Growth Documentation

First, concentrations of viable molds were determined in air, surface, and material samples collected in an office building in Los Angeles, California. The fungal colonies were cultured and identified by microscopic and macroscopic morphology. The levels of culturable fungi via Anderson samples ranged from a high of 40,000 colony-forming units (CFU) for Aspergillus niger to 5,700 CFU for Cladosporium herbarum and to 12 CFU for Penicillium chrysogenum. The results were based upon measurements of 37 different sites of the indoor environment done on multiple dates. They were compared to simultaneously measured outdoor mold counts that were taken at the street and roof levels of the Los Angeles building. The outdoor culturable fungi levels were zero CFU for the A. niger, 265 CFU for the C. herbarum, and zero CFU for the P. chrysogenum.

Study Groups

Eleven patients who were employed in the Los Angeles building were evaluated. The group was composed of four males and seven females, ranging in age from 37 to 64. The ethnic make-up of the group included four Hispanics, four African-Americans, and three Caucasians. The individuals had worked in the building from four to nine years. They reported having a variety of respiratory and pulmonary symptoms, with the most common complaints being sneezing, a runny nose, nasal congestion, shortness of breath, and sinus congestion. The symptoms were related to the work environment since they arose soon after these individuals commenced employment in the building and they lessened when these individuals were away from this jobsite. The primary jobsites of patients were spread throughout different parts of this building. Their patterns of symptoms were not significantly different, which implies that indoor mold contamination is primarily injurious because of its distribution via building heating, ventilation, and air conditioning systems. The mold measurements were obtained so they included multiple portions of the building's ventilation system.

The control group included eleven health volunteers from the same geographic location who matched the age, gender, and ethnicity of the patients. The control subjects had no history of known exposures, either in their worksites or their residences, to mold contamination, and they lacked any related symptoms. None of the control subjects complained of any type of chronic respiratory symptoms, nor had any medical histories of chronic respiratory ailments, either in the past or on an active basis.

Clinical Tests

The eleven patients underwent static pulmonary function testing, methacholine challenge pulmonary testing, and pulmonary exercise testing. Pulmonary function testing was accomplished by measuring the static parameters of forced volume vital capacity (FVC), forced expiratory volume at timed intervals of 1.0 second (FEV-1), FEV-1/FVC ratio, forced expiratory flow (FEF-25%-75%), and diffusing capacity (DLCO). Methacholine and exercise challenge testing were performed according to the American Thoracic Society recommendations [Jones N L, Clinical Exercise Testing. 4th ed. Edn. Philadelphia, 1997; Wasserman K, Hansen, J. E., Sue, D. Y., Whipp, B. J., Casaburi, R., Principles of Exercise Testing and Interpretation. Philadelphia: Lea & Feblger, 1994; and Wassermann K, Pothoff G, Bahra J, Hilger H H. Reversible volume changes of trapped gas in nonspecific bronchoprovocation tests. Chest 1992;101: 970-975.]. The analysis was determined by the achievement of maximal VO₂ (ml/kg/min). The patients also underwent total IgE immunoglobulin analyses, as well as specific RAST analyses for each of the three molds in question.

Mold Cells

Hereafter, “mold” shall include but is not limited to mold, spore, fungi, mycotoxin, or mold-related substance used in a challenge. Mold cells were used in challenges to identify cellular responses and diagnose respiratory disorders.

Mold strains Aspergillus niger, ATCC 16888; Cladosporium herbarum, ATCC 11281; and Penicillium chrysogenum, ATCC 10106 were purchased from the American Tissue Culture Collection (ATCC). Dry-freeze mold samples were suspended in 1.4 ml PBS. The volume of liquid samples were measured and brought to 1.4 ml with PBS. Ten μl of the suspension was loaded into the counting chamber of the Reichert® Bright-Line metalized hemacytometer (Hausser Scientific, Horsham, Pa.), and the cell concentration was determined according to the manufacturer's instructions.

Isolation and Culture of PBMC

PBMC were isolated from blood collected from patients and control subjects as previously described [Gillis B, Gavin I M, Arbieva Z, King S T, Jayaraman S, Prabhakar B S. Identification of human cell responses to benzene and benzene metabolites. Genomics 2007; 90: 324-333 and Gavin I M, Gillis B, Arbieva Z, Prabhakar B S. Identification of human cell responses to hexavalent chromium. Environ Mol Mutagen 2007; 48: 650-657.] and incorporated herein by reference. One milliliter of blood plasma was also collected from the blood samples and stored frozen at −80° C. Cells were adjusted to a concentration of 10⁶ cells/ml in RPMI 1640 medium supplemented with L-glutamine, antibiotics and 10% fetal bovine serum. At least two milliliters of PBMC cultures were placed in tissue culture dishes (Nunc International, Rochester, N.Y.), followed by the addition of mold cells.

Mold cells were added at two concentrations: A. niger at 800 and 4,000 cells/ml, C. herbarum at 2,000 and 10,000 cells/ml and P. chrysogenum at 4,000 and 20,000 cells/ml. Ten μg/ml phytohemagglutinin (PHA-P, Sigma-Aldrich®, St. Louis, MO) was added to separate plates as a positive control. Three replicate plates were used for each mold. Negative control samples contained media only and were used for determining the basal levels of cytokine expression. Plates were placed in a CO₂ water-jacketed incubator (Forma Scientific, Marjetta, Ohio) and incubated for 18 hours.

Cytokine Assay

After overnight culture of the PMBC, one milliliter of supernatant was removed from each plate, centrifuged at 16,000g at +4° C. for 10 minutes, and clear cell-free culture media were collected. Cytokine concentrations in culture supernatants, as well as in plasma, were measured by multiplex immunoassay based on Luminex xMAP™ bead array technology using a Bio-Plex 200 fluorescence bead reader (BioRad Laboratories, Hercules, Calif.). Three panels of antibody-conjugated beads (BioSource, Camarillo, Calif.) for measuring human inflammatory cytokines (GM-CSF, 1L-1β, IL-6, IL-8, TNF-α), Th1/Th2 cytokines (IFN-γ, IL-2, IL-4, IL-5, IL-10) and chemokines (MIP-1α, MIP-1β, MCP-1, Eotaxin, RANTES) were used in the assay according to the manufacturer's instructions.

Statistical Analyses

Statistical analyses were performed with Microsoft Excel and Analyse-it software (Analyse-it Software Ltd., Leeds, UK). An increase of cytokine expression in immunologic challenges was evaluated using a Student's t-test. Statistically significant cytokine responses were then compared between the group of patients and the group of controls using a heteroscedastic Student's t-test. The statistical significance of the difference in responses to various immunologic challenges was evaluated by using a one-way ANOVA test with Tukey type I error correction. The correlation between cytokine response levels and methacholine test results was evaluated by using a non-parametric Spearman correlation test. The confidence level in all tests was set at 5%.

Results

Questionnaire Data

Of the 11 patients, four presented with family histories of asthma, allergies, hayfever, or eczema. Only one of the 11 had any previous medical history of chronic upper or lower respiratory symptoms. Of the 11, one had been an ongoing cigarette smoker, one had smoked before being employed in this job setting and stopped that habit, and one had been a very infrequent smoker (five cigarettes per year) while employed in this job setting.

Regarding the subjective complaints registered, nine out of 11 presented with sneezing, a runny nose, or nasal congestion, which were the most prevalent complaints. The next most common complaint was wheezing, found in eight out of 11. Shortness of breath and coughing was noted in seven. Less frequent complaints included sinus congestion (six of 11), eye itching/burning/tearing (six of 11), and chronic phlegm production (three of 11). The onset of symptoms varied from one to three months after starting in the job environment to more than one year. No discernable pattern was identifiable. None of the eleven reported having any water intrusions, dampness, visible mold, or nasal awareness of mildew-type odors in their personal residences. All 11 patients reported a lessening of their symptoms when away from their jobsites on weekends off or on vacations.

Clinical Data

During the patients' physical examinations, the only abnormalities detectable were signs of rhinitis with inflammation, injection, and edema seen in the nasal mucous membranes. These nasal features were detected in eight out of 11 patients.

Regarding the pulmonary function tests, the FVC measured between 80% and 106% in nine out of 11 patients. Patient 10, a diagnosed asthmatic only after having worked in the building, had a FVC of 70%. Patient 11 had a FVC of 48%, which was consistent with the impact of having smoked daily for 17 years. The FEV-1 measurements in nine out of the 11 ranged from a lot of 89% to a high of 109%. Patient 10 had a FEV-1 of 74% and Patient 11 had a FEV-1 of 49%. The FEV-1/FVC ratio in all 11 ranged from 76% to 96%. The FEV-25%-75% for Patients 1 through 9 ranged from 69% (in a known asthmatic) to a high of 133%. Patient 10 had a 72% result and Patient 11 had a 42% result. The DLCO diffusion testing identified in Patients 1 through 9 ranged from 86% to 123%. Patient 10 had a DLCO of 84% and Patient 11 had a DLCO of 55%.

All of the patients were asked to undergo the methacholine challenge testing, which provokes a narrowing of the airways when the patient breathes in methacholine. A fall in FEV-1 of 20% or greater was deemed a positive test result. The testing first included a challenge with sodium chloride after baseline spirometry had been achieved. Of the 11 patients, nine agreed to have the test. The ongoing cigarette smoker of the group (Patient 11) had spirometric test results too low to permit the test, and one patient who was reported to have a confirmed diagnosis of asthma since working in this environment (Patient 10) asked not to have the test. Of the nine patients who therefore completed the testing, four had a positive response.

All 11 individuals completed the pulmonary exercise testing. The maximal VO2 levels of the patients ranged from a low of 38% to a maximum of 83%, making for a mean of 61.18%. No distinct abnormal patterns were seen through the pulmonary function test modalities, though there clearly were abnormalities in a significant percentage, with the most relevant being an abnormal methacholine challenge test result.

The patients also completed total IgE measurements and RAST-specific IgE antibody analyses. Of the eleven patients, four exceeded the normal range of IgE (0.0-87.0 IU/ml). The overall results of the IgE testing ranged from a low of 1.5 IU/ml to a maximum of 355 IU/ml. The RAST-specific IgE antibody test results for each of the patients was less than 0.35 KU/L regarding the Aspergiullus and Cladosporium, thereby signifying that the results were either absent or undetectable. In one patient, there was an elevated RAST-specific IgE antibody level for Penicillium at 0.47 KU/L, which signified a low level response. Allergy testing found no abnormal patterns, which is not surprising since a damp or moldy environment may contribute to the upper respiratory symptoms even in the absence of IgE-mediated allergic sensitivity [Hope et al., supra].

Each of the patients underwent a complete blood count with a total white cell count and white cell differentials, serum electrolytes and serum chemistries. No abnormalities were identified in those results. When chest x-rays were performed in PA and lateral views, no abnormalities were detected.

These tests of the mold-exposed patients revealed that there were no consistent patterns for detecting any adverse effects when patients were evaluated via static pulmonary function testing, methacholine pulmonary challenge analyses, pulmonary exercise testing, or allergy analyses.

Cytokine Profiling in PMBC Cultures Stimulated with Mold

Cytokines are important mediators of immune response produced in tissues undergoing infection and inflammation. It has been reported that some macrophage and epithelial cell lines produce inflammatory mediators in response to challenges to either mold spores or hyphae [Chiu et al., Overland et al., Huttunen et al., Murtoniemi et al., Shahan et al., supra]. This extensive research therefore investigated (1) if mononuclear cells isolated from peripheral blood were capable of responding to the mold challenges by producing cytokines, and (2) if these responses were affected by previous chronic exposures to mold.

First, PBMC were isolated from blood samples collected from 11 healthy volunteers (who were not constantly exposed to mold) and cultured in the presence of mold cells at two concentrations. Cellular responses were determined by measuring the extracellular expressions of 15 common cytokines: GM-CSF, IFN-γ, IL1-β, IL-2, IL-4, IL-5, IL-6, IL-8, IL-10, TNF-α, MIP1-β, MCP-I, Eotaxin, MIP1-α and RANTES. Extracellular expressions of cytokines to mold were also compared to extracellular expressions of cytokines to phytohemagglutinin (PHA), a lectin from Phaseolus vulgaris (red kidney bean). PHA has high mitogenic activity, induces PMBC proliferation and the simultaneous secretion of cytokines [Leavitt R D, Felsted R L, Bachur N R. Biological and biochemical properties of Phaseolus vulgaris isolectins. J Biol Chem 1977;252: 2961-2966 and Yachnin S, Svenson R H. The immunological and physicochemical properties of mitogenic proteins derived from Phaseolus vulgaris. Immunology 1972;22: 871-883.]. As shown in FIG. 1, mold cells of each of the three types stimulated production of 12 cytokines (GM-CSF, IFN-γ, IL1-β, IL-6, IL-8, IL-10, TNF-α, MIP1-β, MCP-1, Eotaxin, MIP1-α and RANTES), resulting in a statistically significant increase in cytokine concentrations in culture supernatants.

FIG. 1's graphs demonstrate the extracellular expression of cytokines in PBMC cultures of healthy volunteers. The graphs illustrate concentrations in plasma and in culture supernatants of cytokines GM-CSF (A), IFN-γ (B), IL1-β (C), IL-6 (D), IL-8 (E), IL-10 (F), TNF-α (G), MIP1-β (H), MCP-1 (I), Eotaxin (J), MIP1-α (K) and RANTES (L). PBMC from 11 healthy volunteers were challenged with either 1 μg/ml of PHA or mold cells at two concentrations, expressed as thousands of cells per a milliliter of culture. Medium corresponds to unstimulated cell cultures. Numbers in parentheses are the number of samples in which the cytokine concentration was below the lower limit of detection. Stars mark a significant increase in cytokine production compared to unstimulated cultures. The horizontal lines and corresponding numbers denote the lower limit of detection of each cytokine

Three cytokines (IL-2, IL-4, and IL-5) were detected in PHA-stimulated cultures, but were below the detection limit in samples exposed to mold (not shown). The expression levels varied for different cytokines, but concentrations of most cytokines were increased at higher mold doses. There was also a significant sample-to-sample variation in cytokine responses. For many patients, higher responses to PHA correlated with elevated responses to mold challenges, which may reflect the difference in immune competence of PMBC isolated from different subjects.

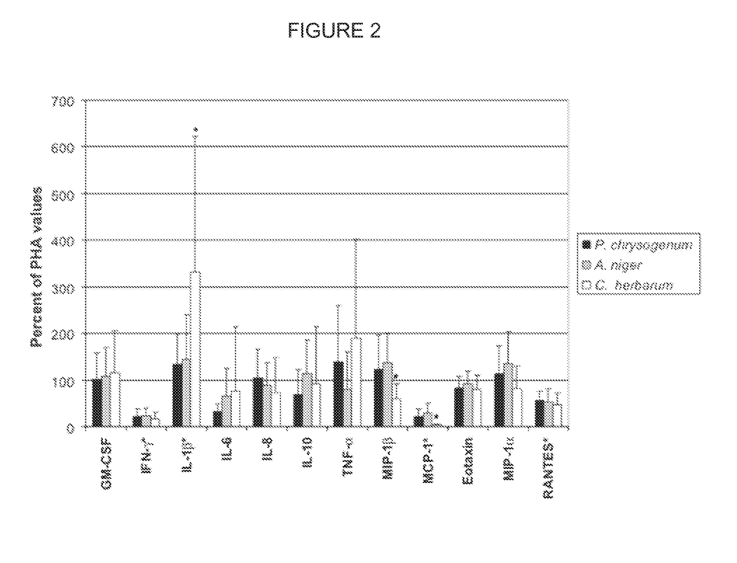

To determine if molds of different types evoke distinct cytokine responses, cellular immunologic responses were compared to various mold challenges and PHA. As shown in FIG. 2, all mold challenges produced a unique pattern of cytokine expression, which was distinct from the PHA-induced pattern. Cytokine concentrations are expressed as percentages of the values in PHA-stimulated cultures. Asterisks mark cytokines (IFN-γ, IL1-β, MCP-1 and RANTES) whose responses to PHA were different from responses to mold challenges by a statistically significant value. Stars mark the responses to mold challenges (IL1-β, MIP1-β, and MCP-1) that are significantly different from the responses to other molds.

In addition to the IL-2, IL-4, and IL-5 responses (detected in PHA-stimulated cultures, but below the detection limit in mold-stimulated cultures), there was an elevated response of inflammatory cytokine IL1-β, and lower responses of cytokine IFN-γ and chemokines MCP-1 and RANTES. When responses to different mold challenges were compared, the responses to A. niger and P. chrysogenum were similar, but distinct from the responses to C. herbarum. There was no increase in MCP-1 expression in cultures challenged with C. herbarum, as there was in other challenges (FIG. 1I). The expression of pro-inflammatory cytokine IL1-β was higher and the expression of the chemokine MIP1-β was lower in the C. herbarum challenge compared to other challenges (FIG. 2). These findings demonstrated that cellular immunologic responses to mold were distinct from responses to mitogens and thus, different types of molds evoke unique patterns of cytokine expression. The expression of cytokines was found to be mold-specific, which may also explain a difference in cytokine responses to other mold species [Grazziutti et al., supra].

Inhibition of Cytokine Responses in Mold-Exposed Individuals

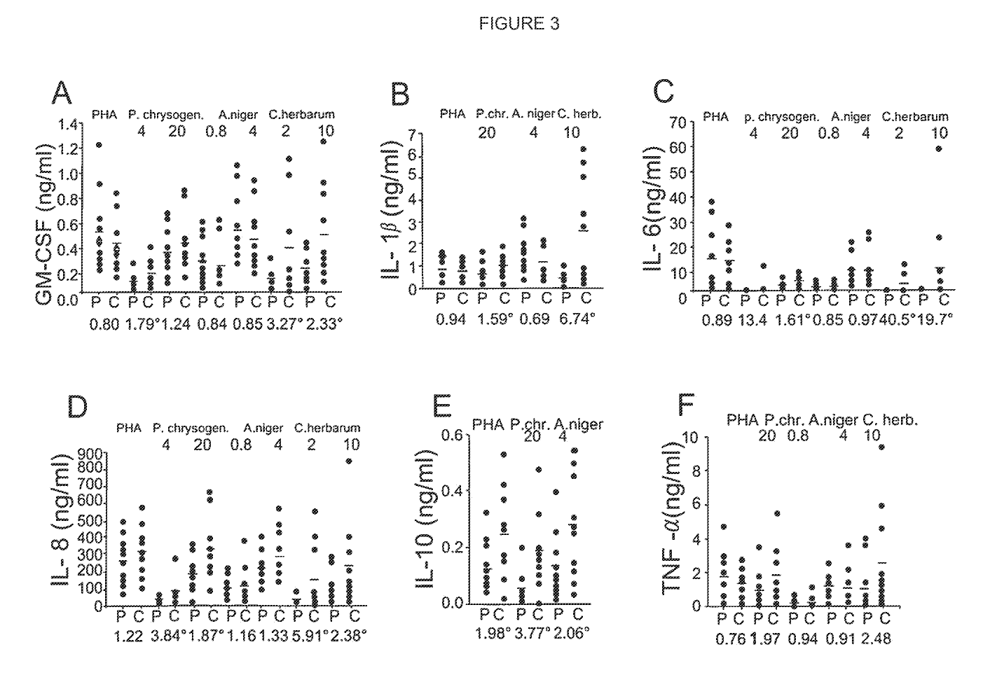

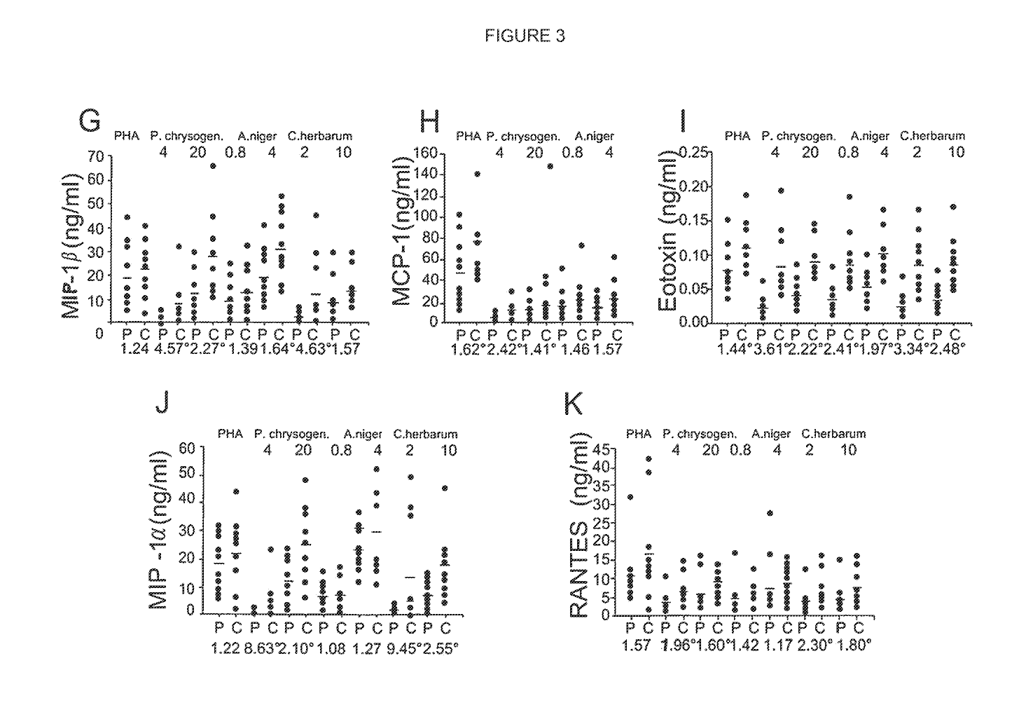

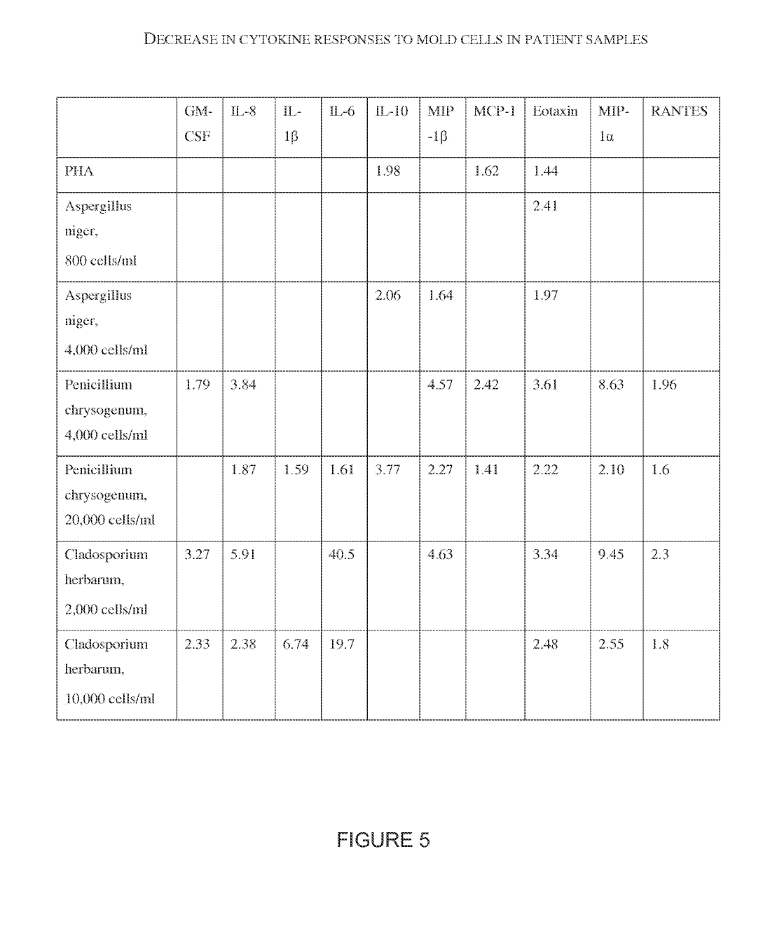

To determine if chronic exposures to mold affected cellular immunologic responses in exposed individuals, cytokine production in patient samples chronically challenged to mold were measured and compared with the responses in control cells. FIG. 3 shows the results of such comparisons for cytokines whose extracellular expression was significantly induced by mold. The statistically significant differences in cytokine expression are summarized in FIG. 5. Numbers represent the ratios of mean cytokine concentrations in the control samples to those in the patient samples.

FIG. 3 demonstrates the inhibition of cytokine responses to mold challenges in patient samples. Cytokine concentrations are shown only for challenges that evoke significant responses in both groups of patients (P) and control subjects (C). Bars represent the mean values in each group. Numbers below the graphs are the ratios of mean control values to mean patient values. Asterisks denote statistically significant differences in patient responses compared to the control values. Individual immunologic challenges are shown above the graphs.

There was a statistically significant decrease in most cytokine responses in patient samples compared to the control cells. A decreased production of ten cytokines (GM-CSF, ILI-β, IL-6, IL-8, IL-10, MIP1-β, MCP-1, Eotaxin, MIP1-α and RANTES) was observed in patient samples challenged with P. chrysogenum. Lower responses of eight cytokines (GM-CSF, IL1-β, IL-6, IL-8, MIP1-β, Eotaxin, MIP1-α and RANTES) were observed in patient samples challenged with C. herbarum. The production of three cytokines (IL-10, MIP1-β, and Eotaxin) was decreased in patient samples challenged with A. niger. These findings demonstrated that chronic exposure to mold affected responses of a unique subset of cytokines in each mold challenge, implicating that cytokine profiling can be used for assessing exposures to individual mold strains. These findings demonstrated for the first time that chronic exposures to mold suppress immune responses to mold challenges in exposed individuals. Such mold challenges can also be used to diagnose respiratory disorders, in particular asthma. In addition to the results discussed herein, the cytokine findings were corroborated by putting every patient through a battery of static, exercise, and methacholine-based pulmonary function tests.

As mentioned earlier, there was a significant variation in immunologic responses of PBMC samples from different individuals (FIG. 1). There is a possibility that attenuated cellular responses to mold in patients resulted from a general decrease in immune competence of the patient cells. To determine if the difference in cellular responses between exposed individuals and control subjects was specific to mold, cellular responses to PHA were compared in two study groups. Cytokine responses to the PHA challenge were similar in both groups, with the exception of IL-10, MCP-1, and Eotaxin (Table 1), which indicates that the observed differences in the expressions of the other seven cytokines were unique to the mold challenges. Lower IL-10 and Eotaxin production was detected in patient samples in all challenges that significantly activate the expressions of these two cytokines, suggesting attenuated IL-10 and Eotaxin responses to both specific and unspecific challenges in exposed individuals. Nevertheless, IL-10 and Eotaxin responses in patient samples were lower in mold challenges, compared to the PHA challenge, which indicates a mold-specific contribution to a decrease of these responses in patient samples.

Correlation of the Suppression of Cytokine Responses with Clinical Symptoms

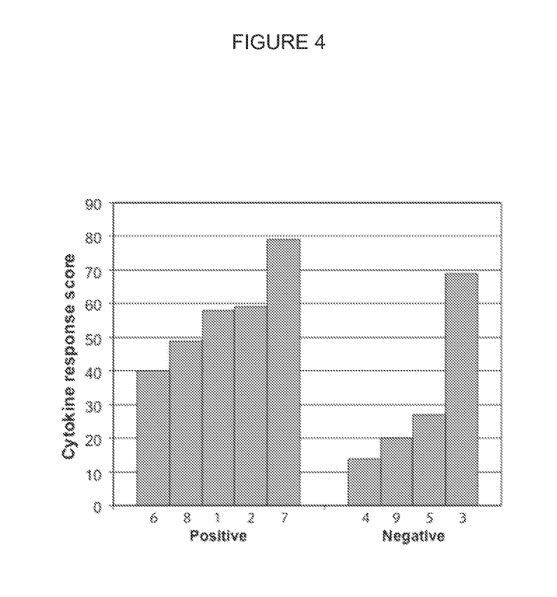

To determine if the observed inhibition of cytokine responses to mold challenges in patients correlates with adverse health effects of chronic exposures to mold, cytokine responses in patient samples was compared with clinical data. For example, a positive methacholine pulmonary function test response signifies hypersensitive or reactive airway disease, which is a frequent byproduct of mold exposure. The only patients who had a positive methacholine response were Patients 3, 4, 5, and 9. The patients were ranked by their cytokine responses to mold challenges, with 1 being the lowest response and 11 being the highest response. The scores for each response were added up for each patient and the resulting values were compared with the methacholine test results (FIG. 4).

FIG. 4 shows the correlation of an inhibition of cytokine response in patients with a positive methacholine challenge pulmonary test response. Cytokine response scores are shown for two groups of patients, who had either a positive methacholine challenge test response (Positive) or a negative pulmonary test response (Negative). Numbers below the graph correspond to the patient codes.

Statistical analysis showed a significant correlation between the positive methacholine test response and an inhibition of cytokine responses to mold challenges. Patients 4, 9, and 5, who had the positive pulmonary test response, had the lowest scores within the patient group. The next lowest score was that of Patient 11, whose spirometric test result was too low to permit the methacholine test. Patient 3 was the only patient who tested positive in the methacholine challenge test, and whose cytokine responses were higher than those of the other patients in the group. These findings demonstrated that the inhibition of cellular responses to mold challenges correlates well with the adverse health effects in mold-exposed individuals, suggesting that cytokine measurements can be utilized in associating clinical symptoms with exposures to mold. Mold challenges can be used for the diagnosis of human respiratory diseases, including asthma. In addition, cytokine measurements can be utilized in detecting exposure to, or inhalation of, mycotoxins. In one embodiment, the method in which this is accomplished is described in para

Determining whether or not there has been exposure is determined by measuring the cytokine levels and determining if they are below or above specified control values by a certain specified factor. The factor depends on the type of cytokine measured and mold used.

One embodiment shown in accordance with the specific invention is shown and described in FIG. 5 throughout the present disclosure. Mycotoxins are toxic chemicals produced by mold. In at least one embodiment of the present disclosure PBMC is challenged with mold cells and mycotoxins were present in PBMC cultures and induced at least part of the observed responses. In at least one embodiment of the present disclosure, the method for measuring exposures to mycotoxins is the same as the method for determining exposures to mold, which is explained and described throughout this disclosure (e.g., [0097], & FIG. 5.

Methods for assaying cytokines at the protein or mRNA levels are well known in the art. Non-limiting examples of method for assaying cytokines at the protein level include enzyme-linked immunoassay (ELISA), Tetramer assay, ELISPOT assay, Fluorospot assay, etc. The cytokines concentration in the plasma, culture supernatant, or cell lysate derived from PBMC can be measured, for example, by multiplex immunoassay based on Luminex xMAP bead array technology, or Bio-Plex 200 fluorescence bead reader (BioRad Laboratories, Hercules, Calif.). In at least one embodiment, the level of one or more cytokine mRNA can be detected (measured) by real time PCR, RT-PCT, Northern blot assay, array hybridization and sequencing, etc. The altered level(s) of the cytokines measured in the affected individual compared to the level from control group is predictive/indicative of FM in the individual. The control group may be healthy individuals in a population and these individuals do not exhibit chronic pain. The altered level of cytokines can be determined using an algorithm and the raw data obtained by measuring the levels of cytokines which have been stored in a computer system, or any other medium that is linked to a computer or machine.

In at least one embodiment, the present disclosure also provides a kit for evaluating an individual afflicted with or at risk of developing FM. The disclosure provides a kit which includes a multiplex of reagents for determining one or more cytokines In one embodiment, the kit includes a multiplex of antibody-coupled beads, each antibody being specific for each of the cytokines to be determined in the assay such as, but not limited to, ELISA, tetramer assay, ELISPOT, Fluorspot, immuno-diffusion, etc. In at least one embodiment of the present disclosure, the kit also provides a detection system. Non-limiting examples of detection systems include one or more radio-labeled, fluorescent-labeled, enzyme-labeled secondary antibody or antibodies. Examples of fluorescent-labels are well known in the art and they include, but are not limited to, fluorescein, Texas red, rhodamine, etc, The bound sample can be analyzed by, for example and not by way of limitation, using a flow cytometer, a fluorescent microscope, a ELISA reader, Luminex xMAP bead array technology, or Bio-Plex 200 fluorescence bead reader.

Mycotoxins are the chemicals secreted by toxic mold. When inhaled, mycotoxins cause the harmful effects associated with toxic mold exposure. Thus, by being able to detect exposure to specific strains of mold, one is able to detect exposure to, or inhalation of, mycotoxins secreted by mold.

Inhibition of cytokine production in mold-sensitized patients may suggest an attempt of controlling the immune response to specific challenges similar to the reduction of the inflammatory responses recently observed in PBMC from allergic sensitized subjects [de Mello L M, Bechara M I, Sole D, Rodrigues V. TH1/TH2 balance in concomitant immediate and delayed-type hypersensitivity diseases. Immunol Lett 2009; 124: 88-94.]. Mold toxins may also suppress immune system as suggested by in vitro studies [Luft P, Oostingh G J, Gruijthuijsen Y, Horejs-Hoeck J, Lehmann I, Duschl A. Patulin influences the expression of Th1/Th2 cytokines by activated peripheral blood mononuclear cells and T cells through depletion of intracellular glutathione. Environ Toxicol 2008;23: 84-95 and Stanzani M, Orciuolo E, Lewis R, Kontoyiannis D P, Martins S L, St John L S, Komanduri K V. Aspergillus fumigatus suppresses the human cellular immune response via gliotoxin-mediated apoptosis of monocytes. Blood 2005;105: 2258-2265.]. Immunosuppressive effects from chronic exposures may explain commonly recorded patient complaints of concurrent susceptibility to infectious organisms and from exposures to chemical irritants. The decrease in cytokine responses correlated well with the severity of the clinical symptoms as was revealed by the medical examination and the methacholine challenge analyses, implying that the suppression of immune responses due to chronic exposures adversely affect the ability of the immune system to fight infections and other environmental challenges.

The mold-specific results were independent of whether the culturable fungi levels in the environment were extremely high or markedly low which demonstrates that adverse human health effects transpire at various concentrations of indoor molds and there is no minimal or unique level that must exist in order to trigger adverse responses and symptoms. As to the implication stated in anecdotal reports concerning the potential injurious effects of molds, there was no pertinence or relevance to any claimed necessary ratio between indoor and outdoor mold counts.

In summary, environmental molds produce a unique cellular effect by stimulating PBMC to release cytokines, which can be determined by cytokine measurements. Therefore, cellular immunologic tests can be used for determining human health effects of exposures to environmental molds. There are objective, reproducible abnormalities that can be linked to specific indoor mold contaminants in regard to human health effects. The identification of these immunologic abnormalities clearly answers why there is a pattern of symptoms and disorders seen by clinicians. They also explain the basis for a concurrent susceptibility to other microorganisms and to chemical substances among such mold-exposed people. Patients do have distinct reactions to individual indoor molds and this can be verified through appropriate immunologic studies. This can be identified irrespective of the specific mold contaminant levels. Concurrent analyses via pulmonary function testing and allergy testing offer only limited insight in documenting these adverse human health effects and should be utilized for additive purposes but they should be recognized for their limited value.

The components, steps, features, objects, benefits and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits and advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The phrase “means for” when used in a claim is intended to and should be interpreted to embrace the corresponding structures and materials that have been described and their equivalents. Similarly, the phrase “step for” when used in a claim embraces the corresponding acts that have been described and their equivalents. The absence of these phrases means that the claim is not intended to and should not be interpreted to be limited to any of the corresponding structures, materials, or acts or to their equivalents.

Nothing that has been stated or illustrated is intended or should be interpreted to cause a dedication of any component, step, feature, object, benefit, advantage, or equivalent to the public, regardless of whether it is recited in the claims.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

BIBLIOGRAPHY

Holt PG, Rowe J, Kusel M, Parsons F, Hollams E M, Bosco A, McKenna K, Subrata L, de Klerk N, Serralha M, Holt B J, Zhang G, Loh R, Ahlstedt S, Sly P D. Toward improved prediction of risk for atopy and asthma among preschoolers: a prospective cohort study. J Allergy Clin Immunol 2010; 125: 653-659, 659 e651-659 e657.

Warrington R J, Whitman C, McPhillips Warrington S. Cytokine synthesis in occupational allergy to caddisflies in hydroelectric plant workers. Int Arch Allergy Immunol 2003;132: 141-147.

Kanda N, Tani K, Enomoto U, Nakai K, Watanabe S. The skin fungus-induced Th1- and Th2-related cytokine, chemokine and prostaglandin E2 production in peripheral blood mononuclear cells from patients with atopic dermatitis and psoriasis vulgaris. Clin Exp Allergy 2002;32: 1243-1250.

Kimura M, Tsuruta S, Yoshida T. Differences in cytokine production by peripheral blood mononuclear cells (PBMC) between patients with atopic dermatitis and bronchial asthma. Clin Exp Immunol 1999;118: 192-196.

1. A method of determining whether a mammal has been exposed to a chemical or biological agent, the method comprising:
(a) exposing the isolated mammalian cells from one or more mammals exposed to and one or more mammals not exposed to a non-toxic amount of a chemical or biological test agent; wherein the chemical or biological test agent is effective to induce a measurable response in the level of a plurality of biomarkers in the isolated mammalian cells from exposed mammals and is not substantially lethal to the survival of the cells;
(b) determining one or more cellular responses to the chemical or biological agent by measuring the level of a plurality of biomarkers of the isolated mammalian cells from the exposed and not exposed mammals;
(c) comparing the cellular responses of the mammals that were exposed to the cellular responses to the mammals not exposed to the chemical or biological agent by determining the difference in the level of the plurality of biomarkers;
(d) generating a biomarker profile for the chemical or biological agent based on the change in the level of a plurality of a subset of the biomarkers; wherein the subset of biomarkers is sufficient to establish a statistically significant correlation between exposure to the agent and the biomarker profile;
(e) isolating mammalian cells from a mammal who is suspected of being exposed to the chemical or biological agent;
(f) determining one or more cellular responses to the chemical or biological agent of the cells by measuring the presence or level of a plurality of biomarkers of the mammalian cells from the mammal suspected of being exposed to the chemical or biological agent; and
(g) determining whether the mammal has been exposed to the chemical or biological agent by determining whether there is a statistically significant correlation between the measured presence or level of the plurality of biomarkers and a biomarker profile generated by measuring the presence or level of a plurality of biomarkers of the mammalian cells from a mammal exposed to the chemical or biological agent.

2. The method of claim 1, wherein the mammalian cells are selected from the group consisting of peripheral blood mononuclear cells, lymphocytes, leukocytes, dendritic cells, epithelial cells, neural cells, and skin cells.

3. The method of claim 1, wherein the cellular responses may be one or more selected from the group consisting of cell proliferation, cell function, cell death, gene expression, protein expression, protein modification, RNA modification, DNA modification, protein function, RNA function, expression of metabolites, protein transfer, RNA transfer, secretion of proteins, and secretion of metabolites.

4. The method of claim 1, wherein the biomarkers may be one or more selected from the group consisting of nucleic acids, proteins, peptides, metabolites, cytokines, chemokines, and growth factors.

5. The method of claim 1, wherein the biomarkers may be one or more cytokines selected from the group consisting of GM-CSF, IL-1βIL-6, IL-8, TNF-α, IFN-γ, IL-2, IL-4, IL-5, IL-10, MIP-1α, MIP-1β, MCP-1, Eotaxin, and RANTES.

6. The method of claim 1, wherein the chemical or biological test agent is selected from the group consisting of industrial chemicals, environmental pollutants, biological toxins, mold, allergens, and antigens.

7. The method of claim 1, wherein the agent may be mold selected from the group consisting of Aspergillus niger, Cladosporium herbarum, and Penicillium chrysogenum.

8. A method of determining whether a mammal has been exposed to a chemical or biological agent, the method comprising:
(a) isolating mammalian cells from a mammal who is suspected of being exposed to the chemical or biological agent;
(b) determining one or more cellular responses to the chemical or biological agent of the cells by measuring the presence or level of a plurality of biomarkers of the mammalian cells from the mammal suspected of being exposed to the chemical or biological agent; and
(c) determining whether the mammal has been exposed to the chemical or biological agent by determining whether there is a statistically significant correlation between the measured presence or level of the plurality of biomarkers and a biomarker profile generated by measuring the presence or level of a plurality of biomarkers of the mammalian cells from a mammal exposed to the chemical or biological agent.

9. The method of claim 8, wherein the mammalian cells are selected from the group consisting of peripheral blood mononuclear cells, lymphocytes, leukocytes, dendritic cells, epithelial cells, neural cells, and skin cells.

10. The method of claim 8, wherein the cellular responses may be one or more selected from the group consisting of cell proliferation, cell death, cell function, gene expression, protein expression, protein modification, RNA modification, DNA modification, protein function, RNA function, expression of metabolites, protein transfer, RNA transfer, secretion of proteins, and secretion of metabolites.

11. The method of claim 8, wherein the biomarkers may be one or more selected from the group consisting of nucleic acids, proteins, peptides, metabolites, cytokines, chemokines, and growth factors.

12. The method of claim 8, wherein the biomarkers may be one or more cytokines selected from the group consisting of GM-CSF, IFN-β, IL1-β, IL-6, IL-8, IL-10, TNF-α, MIP1-β, MCP-1, Eotaxin, MIP1-α and RANTES.

13. The method of claim 8, wherein the chemical or biological test agent is selected from the group consisting of industrial chemicals, environmental pollutants, biological toxins, mold, allergens, and antigens.

14. The method of claim 8, wherein the agent may be mold selected from the group consisting of Aspergillus niger, Cladosporium herbarum, and Penicillium chrysogenum.

15. The method of claim 8, wherein cellular responses to the chemical or biological agent are compared to the cellular responses to a reference agent.

16. The method of claim 15, wherein the reference agent may be selected from the group consisting of lectins and their derivatives, phorbol esters and their derivatives, ionomycin, and its derivatives, lipopolysaccharides and their derivatives, antibodies, and cytokines and their derivatives.

17. A diagnostic kit for measuring a mammal's exposure to a chemical or biological test agent, the kit comprising:
(a) a chemical or biological test agent;
(b) a mammalian cell culture medium,
(c) a balanced salt solution; and
(d) a reference agent.

18. The diagnostic kit of claim 17, wherein the chemical or biological test agent is selected from the group consisting of industrial chemicals, environmental pollutants, biological toxins, mold, allergens, and antigens.

19. The diagnostic kit of claim 17, wherein the mammalian cell culture medium may be RPMI 1640 medium, supplemented with L-glutamine, antibiotics, and 10% fetal bovine albumin.

20. The diagnostic kit of claim 17, wherein the chemical or biological test agent may be mold cells selected from one or more of the group consisting of Aspergillus niger, Cladosporium herbarum, and Penicillium chrysogenum.
